(19) United States
(12) Patent Application Publication
(10) Pub. No.: US 2023/0135837 A1
(43) Pub. Date: May 4, 2023

(54) PROCESS FOR PREPARING A PLATELET LYSATE FRACTION, PLATELET LYSATE FRACTION AND ITS USE FOR TREATING DISORDERS OF THE CENTRAL NERVOUS SYSTEM

(71) Applicants: CENTRE HOSPITALIER REGIONAL ET UNIVERSITAIRE DE LILLE (CHRU), Lille (FR); UNIVERSITE DE LILLE, Lille (FR); UNIVERSITE DU LITTORAL COTE D'OPALE, Dunkerque (FR); INSERM (INSTITUT NATIONAL DE LA SANTE ET DE LA RECHERCHE MEDICALE), Paris (FR); TAIPEI MEDICAL UNIVERSITY, Taipei City (TW)

(72) Inventors: David DEVOS, MARCQ -EN -BAROEUL (FR); Thierry BURNOUF, Lille (FR); Jean-christophe DEVEDJIAN, Lille (FR); Ming-Li CHOU, Taoyuan city (TW); Flore GOUEL, OSTRICOURT (FR)

(21) Appl. No.: 16/650,070

(22) PCT Filed: Sep. 27, 2018
(86) PCT No.: PCT/EP2018/076244

§ 371 Date: Mar. 24, 2020

(30) Foreign Application Priority Data
Sep. 27, 2017 (EP) 17306284.5

(51) Int. Cl. A61K 35/19; C12N 5/078; A61P 25/28

(52) U.S. Cl. CPC A61K 35/19; C12N 5/0644; A61P 25/28

(57) ABSTRACT

The present invention relates on a process for preparing platelet lysate fraction, said process comprising the steps of: 1) providing a platelet lysate, 2) collecting from said platelet lysate a fraction wherein the components exhibit a maximum molecular weight of 100 kDa, on a specific platelet lysate fraction and its use as a drug.

This application is a National Stage Application of PCT/EP2018/076244 filed Sep. 27, 2018, which claims priority from European Patent Application No. 17306284.5 filed Sep. 27, 2017. Each of the prior mentioned applications is hereby incorporated by reference herein in its entirety.

The present invention relates to a process for obtaining a platelet lysate fraction, the platelet lysate fraction itself and its use for treating disorders of the central nervous system such as neurodegenerative, neuroinflammatory, neurodevelopmental and/or neurovascular disorders (i.e strokes), but also the consequences of cerebral insults such as traumatic brain injury or hypoxia.

Developing effective “disease modifying strategy” providing neuroprotection, neurorestoration and neurogenesis to treat neurodegenerative disorders, such as Parkinson's disease (PD), amyotrophic lateral sclerosis (ALS), and Alzheimer disease (AD), is urgently needed considering the huge societal and economic impacts these disorders impose to patients and care-givers.

Developing effective treatments providing neurorestoration and neurogenesis in order to compensate for the loss of neurons and following insults of the central nervous system, such as severe hypoxia following delivery or cardiac arrest or severe traumatic brain injury, is also largely waited considering the lack of validated treatments.

There is substantial evidence that neurotrophins, as activators and modulators of neuronal signaling pathways, represent a logical therapeutic strategy for neurological disorders. Application of single recombinant neurotrophic growth factors has provided encouraging results for neuronal protection and repair in both cell and animal models.

Platelet-derived growth factor-CC (PDGF-CC) proved to be a potent neuroprotective factor in several animal models of neuronal injury whereas PDGF-BB and brain-derived neurotrophic factor (BDNF), administered via intra cerebro-ventricular (ICV) route, stimulated neurogenesis. In addition, systemic administration of BDNF in a photothrombotic model of focal stroke could induce neurogenesis and improve sensorimotor function. Transforming growth factor-β (TGF-β) could promote the development and survival of dopaminergic neurons, and neuroprotection in animal models of parkinsonism, and enhanced the trophic effect of glial-derived neurotrophic factor (GDNF) in hemiparkinsonian rats¹.

Pre-clinical studies showed neuroprotection by basic-fibroblast growth factor (b-FGF) and vascular endothelial growth factor-β (VEGF-β), and promotion of neuroprotection and neurorestoration by GDNF. Unfortunately, all randomized clinical studies involving ICV administration of high-dose, single growth factors have failed to yield any substantial positive clinical effects. Currently, administering single neurotrophins in such complex and multifaceted neurodegenerative pathologies is insufficient to yield meaningful therapeutic outcomes.

Thus, there is a need to develop a novel approach which would likely be more powerful, safe to use and easy to produce, but it is conceptually challenging in particular to seek regulatory approval, thereby justifying more pragmatic strategies inspired from other fields of regenerative medicine.

Platelet concentrates are well-established therapeutic product, on the WHO model list of essential medicines, typically used in the prophylaxis and treatment of bleeding disorders resulting from thrombocytopenia. Besides their role in haemostasis, platelets exert crucial physiological functions in wound healing and tissue repair.²

The range of regenerative medicine³ and cell therapy⁴ applications where platelets and platelet lysates are evaluated is expanding. The therapeutic benefit of platelets in tissue healing is multifactorial and results from the myriad of bioactive mediators stored primarily in the α-granules and acting in synergy. These include neurotrophic growth factors, such as PDGF (-AA, -AB and -BB isoforms), BDNF, VEGF, TGF-β, bFGF, or epithelium growth factor (EGF). Intracranial delivery of platelet lysates in animal models of stroke was recently shown to stimulate the proliferation of endogenous neural stem cells (eNSC) and angiogenesis in the subventricular zone and in the pen-lesion cortex, leading to improved functional outcomes and reduced injury, and suggesting neuroprotective effects⁵.

Moreover, platelet lysates contain a huge pool of molecules and compounds which are not fully characterized, but it is known that platelet lysate contains plasma-borne fibrinogen, a protein that plays a causative role in neurologic disorders as a potent inducer of inflammation and an inhibitor of neurite outgrowth. This may be the reasons why application of platelet lysate, or a platelet lysate derived product, in the field of disorders of the central nervous system in humans, such as Parkinson's disease or amyotrophic lateral sclerosis, has not been reported yet.

Thus, it is to the credit of the Applicants to have been able to discover a novel platelet lysate derived product, useful in particular in treating disorders of the central nervous system, and exhibiting properties which have not been identified yet. In particular, Applicant have succeeded to obtain different platelet lysate fractions which exhibit a strong neuroprotective effect.

In a first aspect, the present invention relates to a process for preparing a platelet lysate fraction, said process comprising the steps of:
1) providing a platelet lysate,
2) collecting from said platelet lysate a platelet lysate fraction wherein the components exhibit a maximum molecular weight of 100 kDa.

According to the invention, the first step of the process consists in providing a platelet lysate. This platelet lysate may be a platelet pellet lysate (PPL) or a pooled human platelet lysate (pHPL). Preferably, the platelet lysate is a pooled human platelet lysate (pHPL).

Both PPL and pHPL may be prepared according to well-known methods from platelet concentrate (PC), which induce the release of growth factors and other active molecules.

In a first embodiment, the platelet lysate provided in step 1) is a platelet pellet lysate (PPL). The PPL may be prepared as described in the art⁶. It may for example be prepared as follows:
i) Providing a platelet concentrate (PC),
ii) Centrifuging said platelet concentrate so as to obtain a platelet pellet and a first supernatant,
iii) Removing the supernatant and suspending the pellet in a physiological buffer,
iv) Freeze-thawing said suspended pellet,
v) Centrifuging the suspension obtained in step iv) so as to obtain a platelet pellet lysate and second supernatant.

The platelet concentrate provided in step i) may be obtained by suitable standard collection methods from autologous or allogeneic platelet sources, in particular from whole blood, or by apheresis procedures, and suspended in plasma, or a combination of plasma and platelet additive solution, or platelet additive solution only⁷. Moreover, the platelet concentrate may be leukoreduced.

Suitable physiological buffers used in step iii) are for example phosphate buffer saline (PBS), HEPES buffer, Tris-HCl buffer or sodium acetate buffer, or physiological saline.

The platelet pellet lysate (PPL) may be fresh PPL (PPL^{F}) or expired PPL (PPL^{E}), preferably PPL^{F}. The term fresh PPL refers to platelet pellet lysate prepared from platelet concentrates processed within 5 days of collection (non-expired). The term expired PPL refers to platelet pellet lysate prepared from platelet concentrates processed over 5 days of storage.

According to a second embodiment, the platelet lysate provided in step 1) is a pooled human platelet lysate (pHPL). For example, the pHPL may be prepared by the method comprising the following steps of:
a) providing platelet concentrates,
b) lysing separately each platelet concentrate of step a), and
c) mixing the lysates resulting from step b) in order to obtain a pooled human platelet lysate.

The platelet concentrates provided in step a) may come from different donors and may be obtained by suitable standard collection methods from allogeneic platelet sources. Particularly, the platelet concentrate may be obtained from whole blood using the buffy coat or platelet-rich plasma (PRP) technique, or may be collected by apheresis technique. Preferably, the platelet concentrate is produced from whole blood using the buffy coat or (PRP) technique⁸.

In the “PRP method”, anticoagulated whole blood is centrifuged using a soft spin under conditions validated to segregate red blood cells (RBC) from the upper half containing a platelet and plasma mixture, so called PRP. Platelets are then concentrated by hard spin centrifugation with validated acceleration and deceleration curves. The platelet concentrate bag is left stationary at room temperature and then the concentrate is resuspended in plasma. In the “buffy coat” method, anticoagulated whole blood is centrifuged using a hard spin with validated acceleration and deceleration curves to separate ‘cell-free” plasma on the top layer, a middle layer called buffy coat (BC) and a red blood cells (RBC) bottom layer. The BC layer is transferred to a satellite bag. A small quantity of plasma is returned to the BC layer and gently mixed before again being subjected to light spinning centrifugation with validated acceleration and deceleration curves. The PRP supernatant is then placed in platelet storage and may be stored at 22+/−2° C.

In the apheresis method, the platelet concentrates may be obtained through an extracorporeal medical device used in blood donation that separates the platelets and returns other portions of the blood to the donor.

The plasma used for suspending the concentrate in the “PRP method”, the plasma returned to BC layer in the “buffy coat” method, or the plasma collected with platelet by apheresis may be substituted by a platelet additive solution (PAS) or by a mixture between plasma and PAS, and preferably by a mixture between plasma and PAS. Said mixture between plasma and PAS may contain from about 30% to 40% by weight of plasma and from about 70% to 60% by weight of PAS.

The platelet concentrate provided in step a) may be subjected to a leucodepletion treatment. This treatment leads to leucocyte depletion and it may be achieved by filtration on a leucoreduction filter or during the platelet collection by apheresis.

The platelet concentrate provided in step a) may be subjected to a step of viral/pathogen inactivation treatment before lysis. The viral/pathogen inactivation treatment applied to the platelet concentrate may be selected from Intercept® Blood system (from Cerus Corporation), Mirasol® PRT system (from Terumo BCT), or THERAFLEX-UV (from Macopharma). These procedures are well-known by one skilled in the art and target, with or without the addition of a photo-inactivating agent, the alteration of nucleic acids.

The platelet concentrate may also be subjected to a leucodepletion treatment and to a viral/pathogen inactivation treatment. Preferably, the leucodepletion treatment is performed before the viral/pathogen inactivation treatment.

The step b) of lysing separately each platelet concentrates may be achieved by any method known in the art. For example, platelet lysis may be achieved by one or more freeze/thaw cycles, by platelet activation induced by addition of thrombin or CaCl₂, by sonication or by solvent/detergent (S/D) treatment. Preferably, step b) of lysis is achieved by one or more freeze/thaw cycles, and more preferably by at least three cycles. When lysis is achieved by one of the preceding method, a centrifugation and filtration step may also be performed to remove cell debris.

Then, step c) consists in mixing the lysates in order to obtain a pooled human platelet lysate, also called pHPL. Thus, the pool of HPL is obtained by mixing the lysed platelet concentrates from at least 2 platelet lysates from different donors. Preferably, the pool of HPL is obtained by mixing at least 5, at least 10, at least 20, at least 30, at least 40, at least 50, at least 100 at least 140, at least 180, at least 200 and more particularly, at least 240 different platelet lysates collected from different donors.

A suitable pooled human platelet lysate (pHPL) for the process of the invention may be any pooled human platelet lysate from blood establishments or from commercial suppliers. For example, the pooled human platelet lysate may be obtained from Macopharma (Tourcoing, France; MultiPL'30® Human platelet lysate), from Cook-Regentec (Indianapolis, USA; Stemulate ® Human platelet lysate), from Stemcell Technologies (Grenoble, France; Human platelet Lysate) or also from Sigma-Aldrich (PLTMax® Human Platelet Lysate).

According to this second embodiment, the pHPL may be subjected to a treatment which induces an activation of the coagulation cascade. For example, the pHPL may be mixed with glass beads (GB) and CaCl₂ under stirring, or using CaCl₂ alone. This treatment leads to a clot formation that is removed after centrifugation and the resulting pHPL is thus free of fibrinogen. Without wanting to be bound by any theory, the inventors believe that this treatment contributes to lower toxicity and improved neuroprotective effect of the obtained platelet lysate fraction according to the invention.

The second step of the process consists in collecting a platelet lysate fraction wherein the components exhibit a maximum molecular weight of 100 kDa. Hence, the collected heat-treated platelet lysate fraction may not contain components with a molecular weight greater than 100 kDa.

In the meaning of the present invention, a platelet lysate fraction wherein the components exhibit a maximum molecular weight of X kDa is called “platelet lysate X kDa fraction” or also “X kDa fraction”, e.g “platelet lysate 100 kDa fraction” or “100 kDa fraction”.

Particularly, the collection step is performed in order to obtain a platelet lysate fraction wherein components exhibit a maximum molecular weight of 100 kDa, of 90 kDa, of 80 kDa, of 70 kDa, of 60 kDa, of 50 kDa, of 40 kDa, of 30 kDa, of 20 kDa, of 10 kDa, of 5 kDa, of 3 kDa or of 1 kDa. In other terms, the platelet lysate fraction may not contain components with a molecular weight greater than 100 kDa, 90 kDa, 80 kDa, 70 kDa, 60 kDa, 50 kDa, 40 kDa, 30 kDa, 20 kDa, 10 kDa, 5 kDa, 3 kDa, or greater than 1 kDa.

According to this second step, the so-obtained fractions are preferably the platelet lysate 50 kDa fraction, the platelet lysate 30 kDa fraction, the platelet lysate 20 kDa fraction, the platelet lysate 10 kDa fraction and the platelet lysate 3 kDa fraction, more preferably the platelet lysate 10 kDa fraction and the platelet lysate 3 kDa fraction, and even more preferably the platelet lysate 3 kDa fraction.

Surprisingly and unexpectedly, the inventors have found that the platelet lysate fraction according to the invention exhibits a strong neuroprotective activity although the platelet lysate was subjected to a collection step in order to remove components. Indeed, the so-obtained fractions still exert a neuroprotective effect while it was expected that the removing of some components according to their molecular weight would lead to the loss of said effect. Still surprisingly, the neuroprotective activity is obtained for the smallest fractions for which it was believed that the lack of components with high molecular weight would have been detrimental for the neuroprotective activity.

Thus, the present invention represents a major breakthrough to provide an alternative treatment of central nervous system disorders.

The collection step may be performed by any method known in the art which leads to the separation and/or concentration of components contained in a liquid according to their molecular weight.

In one embodiment, the collection step may consist in fractionating the supernatant obtained by centrifugation of the platelet lysate in order to collect the so-called platelet lysate 100 kDa fraction. In this embodiment, the fractionation may be performed by ultrafiltration. According to this ultrafiltration method, a centrifugal filter with vertical membrane having a 100 kDa cut-off may be used. The centrifugal filter is thus filled with the supernatant obtained after the third step and is subjected to the centrifugation. The angle rotor, the spin speed and the spin time may be determined by one skilled in the art. Moreover, one skilled in the art may adapt the cut-off used in order to obtained the desired platelet lysate fraction according to the invention.

The platelet lysate fraction of the invention has a reduced protein content. By the expression “reduced protein content”, means that said fraction contains less than 1.5 μg/μL of proteins, preferably less than 1.0 μg/μL, and more preferably less than 0.70 μg/μL. The protein content may be determined by any method known in the art and for example by Lowry protein assay or by ELISA.

Specifically, the platelet lysate 3 kDa fraction may present a protein content of about 0.05 μg/μL to about 0.30 μg/μL, particularly of about 0.05 μg/μL to about 0.30 μg/μL, and more particularly a protein content of about 0.05 μg/μL to about 0.1 μg/μL. Moreover, the platelet lysate 3 kDa fraction is preferably free of fibrinogen and free of growth factors.

The process of the invention may comprise an additional step of storing the heat-treated platelet lysate fraction at −80° C. for further use.

In one preferred embodiment, before the collection step, the process of the invention may also comprises a step of heat-treating the platelet lysate at a temperature of about 50° C. to about 70° C. during 15 minutes to 45 minutes, and a step of centrifuging said heat-treated platelet lysate and keeping the supernatant.

The heat-treatment step is preferably performed without adding stabilizers that are classically used to maintain the biologic activity of protein. Such stabilizers are for examples sucrose, sorbitol, mannitol or amino acids such as arginine or lysine.

It is believed that the heat-treatment step induces precipitation of some proteins which are thus removed after the centrifuging step. A reduced content in protein may be more advantageous for some applications, for example in the treatment of a disorder of the central nervous system through an intranasal administration.

The heat-treatment step may be performed at a temperature of about 50° C. to about 70° C., preferably at a temperature of about 50° C. to about 60° C., and more preferably at a temperature of about 54° C. to 58° C. The heat-treatment step is for example performed at 56° C.

The duration of the heat-treatment step may be from 15 to 45 min, preferably from 20 to 40 minutes, and more preferably from 25 to 35 minutes. For example, the heat-treatment step is performed for 30 minutes.

The centrifugation may advantageously be carried out at a temperature of about 2° C. to 6° C. The duration of this centrifugation step is at least 10 minutes and the speed may be of about 8000×g to about 12000×g, preferably of about 9000×g to about 11000×g, and more preferably around 10000×g. The supernatant is recovered and used for the collection step of the process.

According to this embodiment, the resulting platelet lysate fraction after the collection step is a heat-treated platelet lysate fraction.

It has been surprisingly and unexpectedly found that heat-treated platelet lysate fractions according to this embodiment exhibit a strong effect in terms of neuroprotection although the platelet lysate was subjected to a heat-treatment step and to a collection step.

Moreover, In vitro assays have shown that the fractions prepared according to the process of the invention, with or without a step of heat-treatment, protect dopaminergic cells from death induced by a neurotoxin. Without wanting to be bound by any theory, the inventors believe that the improved neuroprotective activity of the fractions is a result of their reduced protein content, such as fibrinogen content, and the result of the concentration of compounds with a molecular weight not greater than 100 kDa, and particularly, not greater than 50 kDa, 30 kDa, 20 kDa, 10 kDa or 3 kDa. The results obtained in vitro have been confirmed with in vivo assays on a well-known model of amyotrophic lateral sclerosis (ALS) which is a transgenic mice overexpressing mutant forms of the copper/zinc superoxide dismutase gene.

Particularly, it is also believed that the collection step, and optionally the heat-treatment, result in reduction or depletion of fibrinogen and proteolytic enzymes (such as thrombin, or thrombin-like, or thrombin-generating coagulation factors), and particularly that the heat-treatment step precipitates and/or inactivates potentially toxic heat-unstable proteins and favorably modifies the protein and growth factor balance in the resulting fraction as well as the collection which modifies the component's molecular weight balance and potentiate the neuroprotective effect. Thus, the resulting platelet lysate fraction may avoid the biological risk of fibrin formation, which is toxic for the brain.

Therefore, the obtained platelet lysate fraction according to the invention offers a substantially higher safety margin than standard human platelet lysates suspended in plasma and is more suitable and more efficient for use in biotherapy, especially through brain administration.

As set forth above, platelet lysate fractions obtained by the process of the invention provides improved neuroprotective activity.

In a second aspect, the invention relates to a platelet lysate fraction. The platelet lysate fraction may be obtained according to the method described above. Hence, the platelet lysate fraction according to the invention is specifically a platelet fraction wherein the components exhibit a maximum molecular weight of 100 kDa.

Particularly, the platelet lysate fraction according to the invention is a platelet lysate 50 kDa fraction, a platelet lysate 30 kDa fraction, a platelet lysate 20 kDa fraction, a platelet lysate 10 kDa fraction or a platelet lysate 3 kDa fraction. More preferably, the fraction according to the invention is a platelet lysate 10 kDa fraction or a platelet lysate 3 kDa fraction, and even more preferably a heat-treated platelet lysate 3 kDa fraction.

The platelet lysate fraction of the invention has a reduced content in proteins. By the expression “reduced content” it is meant that said fraction contains less than 1.5 μg/μL of proteins, preferably less than 1.0 μg/μL and more preferably less than 0.70 μg/μL.

Specifically, the platelet lysate 3 kDa fraction may present a protein content of about 0.05 μg/μL to about 0.30 μg/μL, particularly of about 0.05 μg/μL to about 0.30 μg/μL, and more particularly a protein content of about 0.05 μg/μL to about 0.1 μg/μL. Moreover, the heat-treated platelet lysate 3 kDa fraction is preferably free of fibrinogen.

The platelet lysate fraction may be obtained by the process described hereabove.

The platelet lysate fractions according to the invention display a strong neuroprotective activity and are particularly advantageous for treating disorders of the central nervous system. More particularly, the smallest platelet lysate fractions, i.e the platelet lysate 10 kDa fraction and the platelet lysate 3 kDa fraction, can more easily pass through the nasal cavity in order to penetrate into the brain and exert the neuroprotective effect.

In a third aspect, the invention relates to the platelet lysate fraction according to the invention for use as a biological drug or “biotherapy”.

Indeed, thanks to its improved neuroprotective activity and its higher safety, platelet lysate fraction may be used in the treatment and/or prevention of a disorder of the central nervous system.

In other terms, the invention also relates to a method of treating and/or preventing disorders of the central nervous system, comprising the administration of a therapeutically effective amount of the platelet lysate fraction of the invention, to a patient in need thereof. Preferably the patient is a warm-blooded animal, more preferably a human.

Disorders of the central nervous system within the meaning of present invention include but are not limited to neurodegenerative disorders, neurovascular disorders, neuroinflammatory disorders, neurodevelopmental disorders such as autism and schizophrenia, cerebral insult such as severe hypoxia following delivery or cardiac arrest or severe cranial traumatism/traumatic brain injury that is to say severe insults resulting in a significant loss of neurons leading to handicap.

In a preferred embodiment, the disorder of the central nervous system is a neurodegenerative disorder. Neurodegenerative disorders within the meaning of the present invention include, but are not limited to multiple sclerosis (MS), Parkinson's disease (PD), Huntington's disease (HD), Amyotrophic lateral sclerosis (ALS), stroke, age-related macular degeneration (AMD), degenerative diseases of the retina, and dementia, the latter including, without being limited thereto, Alzheimer's disease (AD), vascular dementia, frontotemporal dementia, semantic dementia and dementia with Lewy bodies. Preferably, the neurodegenerative disorder is selected from Alzheimer's disease, Parkinson's disease, Huntington's disease, multiple sclerosis and Amyotrophic lateral sclerosis, more preferably from Parkinson's disease and Amyotrophic lateral sclerosis.

In another embodiment, the disorder of the central nervous system is a cerebral insult of the central nervous system such as severe hypoxia following delivery or cardiac arrest or severe cranial traumatism that is to say severe insults resulting in a significant loss of neurons leading to handicap. The early treatment, with the platelet lysate fraction, following the insult may enhance the physiological neurorestoration and neurogenesis abilities.

The platelet lysate fraction may be administered as such, be encapsulated in natural or synthetic nanoparticles⁹ or microparticles or be comprised in a pharmaceutical solution further comprising at least one pharmaceutically acceptable carrier, diluent, excipient and/or adjuvant. The pharmaceutical solution can further comprise complexes, molecules, peptides, salts, vectors or any other compound, which can ameliorate or can be beneficial in treatment neurological disorders.

The route of administration and the dosage regimen naturally depend upon the severity of the illness, the age, weight, and sex of the patient, etc. The platelet lysate fraction of the invention may be used for the treatment of any patient, especially a warm-blooded animal such as a mammal and preferably a human.

Advantageously, the platelet lysate fraction according to the invention is suitable for the administration in the central nervous system. Specifically, said platelet lysate fraction is adapted for intranasal (e.g. Parkinson's disease which is a pathology of the substantia nigra, striatum and olfactory bulbar close to the nasal cavities) or intra thecal (e.g. for amyotrophic lateral sclerosis which is a pathology of the spinal cord) or intra cerebroventricular (ICV) administration, preferably closed to the intraventricular foramen so that the platelet lysate fraction can be administrated into the third ventricle.

As the smallest platelet lysate fractions still exhibit a neuroprotective activity, they are particularly effective for an intranasal administration. Indeed, thanks to their low molecular weight components, those fractions can easily penetrate the brain through the nasal cavity which is advantageous for the purpose of the invention in order to treat disorders of the central nervous system. Moreover, in terms of safety, it is more advantageous for the patient to get benefit from neuroprotective effect with a platelet lysate which contain less components.

Administration to the central nervous system may be achieved by the method known in the art. For example, administration may be carried out with a drug delivery system, such as a programmable medication pump.

The administration of the platelet lysate fraction of the invention may also be performed by any other method known by the person skilled in the art, such as for example, intraveneous, intraperitoneal, intramuscular or intraocular administration, or perfusion or infusion of an organ (i.e. direct infusion of a part of the brain tissue).

The exposure dosage used for the administration may be adapted as a function of various parameters, and in particular as a function of the mode of administration used, of the relevant pathology or of the desired duration of treatment.

Definitions

The definition and explanations below are for the terms as used throughout the entire application, including both the specification and the claims.

By “neuroprotective activity” or “neuroprotection” is meant preservation of neuronal structure and/or function of neuronal cells affected by neurotoxin compared to neuronal cells, which are not affected by neurotoxin. Neuroprotection aims to prevent or slow the disease progression and secondary injuries by halting or at least slowing the loss of neurons. For example, it refers to preservation of the number of neurons in the striatum and/or in the substantia nigra pars compacta of patients affected by Parkinson's disease compared to patients who are not affected by Parkinson's disease.

By “neurorestoration” is meant compensation of existing alterations and stimulation of structural and functional restoring of the injured nervous activity.

The term “patient” refers to a warm-blooded animal, more preferably a human, who/which is awaiting or receiving medical care or is or will be the object of a medical procedure.

The term “human” refers to subjects of both genders and at any stage of development (i.e. neonate, infant, juvenile, adolescent, adult). In one embodiment, the human is an adolescent or adult, preferably an adult.

The terms “treat”, “treating” and “treatment”, as used herein, are meant to include alleviating or abrogating a condition or disease and/or its attendant symptoms.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “therapeutically effective amount” (or more simply an “effective amount”) as used herein means the amount of platelet lysate fraction of the invention which is sufficient to achieve the desired therapeutic or prophylactic effect in the individual to which it is administered.

The term “administration”, or a variant thereof (e.g., “administering”), means providing the platelet lysate fraction of the invention, alone or as part of a pharmaceutically acceptable solution, to the patient in whom/which the condition, symptom, or disorder is to be treated or prevented.

The present invention will be better understood with reference to the following examples and figures. These examples are intended to be representative of specific embodiments of the invention, and are not intended as limiting the scope of the protection.

### FIGURES

FIG. 1: Time course of treatment with platelet lysate and platelet lysate fractions. Platelet lysate (control) and fractions were added 1 h before erastin (A) or 1 h, 3 h, 6 h and 8 h after erastin (B).

FIG. 2: Neuroprotective effect measured by cytometry of 50 kDa, H-50 kDa, 30 kDa, H-30 kDa, 10 kDa, H-10 kDa, 3 kDa and H-3 kDa fractions. The viability was measured by propidium iodide assay and normalized to the control (non treated cells) +/−SEM (n=1 for 50 kDa, 30 kDa, 10 kDa and n=2 for 3 kDa).

FIG. 3: Neuroprotective effect of 50 kDa, H-50 kDa, 30 kDa, H-30 kDa, 10 kDa, and H-10 kDa fractions measured by resazurin. Treatment 1 h before erastin (E). The viability was measured and normalized to the control (non treated cells) n=1.

FIG. 4: Involved pathway and neuroprotective effect of 3 kDa and H-3 kDa fractions measured by resazurin. Treatment 1 h before erastin (E). iAkt: Akt inhibitor, E: Erastin. The viability was measured and normalized to the control (non treated cells) +/−SEM (n=4).

FIG. 5: Neuroprotective effect of 3 kDa and H-3 kDa fractions measured by resazurin. Treatment of LUHMES cells 1 h, 3 h, 6 h or 8 h after Erastin (E).

FIG. 6: Neuroprotective effect measured by cytometric assay of H-3 kDa fraction. Treatment 1 h before Erastin (E). The viability was measured and normalized to the control (non treated cells) +/−SEM (n=4 for pHPL, H-pHPL, H-pHPL-GB, and n=2 for H-3 kDa

FIG. 7A: Body weight evolution of male mice treated by vehicle and H-PPL diluted preparation. Male WT: Male wild-type, Male Tg: male FVB-Tg (Sod1*G86R), Veh:Vehicle.

FIG. 7B: Body weight evolution of female mice treated by vehicle and H-PPL diluted preparation. Female WT: Female wild-type, Female Tg: Female FVB-Tg (Sod1*G86R), Veh:Vehicle.

FIG. 8: Survival curve of male and female mice treated by vehicle and H-PPL diluted preparation. Male Tg: male FVB-Tg (Sod1*G86R), Female Tg: Female FVB-Tg (Sod1*G86R).

FIG. 9A: Body weight evolution of male mice treated three times a week by vehicle and H-3 kDa preparation. Male WT: Male wild-type, Male Tg: male FVB-Tg (Sod1*G86R), Veh:Vehicle.

FIG. 9B: Body weight evolution of female mice treated three times a week by vehicle and H-3 kDa preparation. Female WT: Female wild-type, Female Tg: Female FVB-Tg (Sod1*G86R), Veh:Vehicle.

FIG. 10: Survival curve of male and female mice treated three times a week by vehicle and H-3 kDa preparation. Male Tg: male FVB-Tg (Sod1*G86R), Female Tg: Female FVB-Tg (Sod1*G86R).

FIG. 11A: Body weight evolution of male mice treated six times a week by vehicle and H-3 kDa preparation. Male WT: Male wild-type, Male Tg: male FVB-Tg (Sod1*G86R), Veh:Vehicle.

FIG. 11B: Body weight evolution of female mice treated six times a week by vehicle and H-3 kDa preparation. Female WT: Female wild-type, Female Tg: Female FVB-Tg (Sod1*G86R), Veh:Vehicle.

FIG. 12: Survival curve of male and female mice treated six times a week by vehicle and H-3 kDa preparation. Male Tg: male FVB-Tg (Sod1*G86R), Female Tg: Female FVB-Tg (Sod1*G86R).

### EXAMPLES

The following abbreviations are used throughout the entire description, figures and claims:

10 kDa fraction: platelet lysate 10 kDa fraction

30 kDa fraction: platelet lysate 30 kDa fraction

3 kDa fraction: platelet lysate 3 kDa fraction

50 kDa fraction: platelet lysate 50 kDa fraction

H-10 kDa fraction: heat-treated platelet lysate 10 kDa fraction

H-30 kDa fraction: heat-treated platelet lysate 30 kDa fraction

H-3 kDa fraction: heat-treated platelet lysate 3 kDa fraction

H-50 kDa fraction: heat-treated platelet lysate 50 kDa fraction

H-pHPL: heat-treated pooled human platelet lysate

H-pHPL-GB: pooled human platelet lysate mixed with glass beads (GB) and subjected to heat-treatment

HPL: human platelet lysate

H-PPL: heat-treated platelet pellet lysate

ICV: intra cerebro-ventricular

PAS: platelet additive solution

PBS: phosphate buffer saline

PC: platelet concentrate

pHPL: pooled human platelet lysate

PL: platelet lysate

PPL: platelet pellet lysate

PPL^{E}: platelet pellet lysate from expired PC

PPL^{F}: platelet pellet lysate from non-expired PC

PRP: platelet-rich plasma

RBC: red blood cells

### Example 1—Experiments with Platelet Pellet Lysate (PPL) as Starting Platelet Lysate Material

Materials and Methods

1. Preparation of Platelet Pellet Lysate and Platelet Lysate Fractions

Platelet lysates were obtained from platelet concentrates (Etablissement Francais du Sang, Lille, France). After centrifugation at 4600×g for 20 minutes at room temperature, platelet pellets were washed twice and resuspended in PBS in 1/10th of the initial volume. Then, platelet pellets were frozen (nitrogen) and thawed (37° C.) three times, and centrifuged at 4600×g for 20 minutes at room temperature.

The supernatant, called “Platelet Pellet Lysate” (PPL), was collected, aliquoted and stored at −80° C.

One part of the PPL was heat-treated at 56° C. for 30 minutes, then centrifuged at 10000×g for 15 minutes at 4° C. and the supernatant, called “Heat-treated Platelet Pellet Lysate” (H-PPL), was aliquoted and stored at −80° C.

Platelet lysate fractions were obtained from PPL and H-PPL, by performing the fractionation step using Amicon Ultra −0.5 ultrafiltration tubes including different cutoffs (Amicon Ultra-0.5 centrifugal Fitter Devices, Millipore).

Briefly, 500 μL of PPL or H-PPL is added to the filter device inserted into the collect tube, and centrifuged at 14000×g for 30 minutes at 4° C. with a fixed angle rotor at 40°. According to the cutoff used, the filtrate or the platelet lysate fraction, which is the lower part lower than the cutoff, is called 50 kDa fraction, 30 kDa fraction, 10 kDa fraction and 3 kDa fraction when obtained from PPL, and called H-50 kDa fraction, H-30 kDa fraction, H-10 kDa fraction and H-3 kDa fraction when obtained from H-PPL.

The different platelet lysate fractions were then aliquoted and stored at −80° C. for further experiments.

2. LUHMES Cells Maintenance and Differentiation

LUHMES cells were obtained from Dr. Scholz's laboratory (University of Konstanz, Germany) and cultured as described¹⁰

Briefly, undifferentiated LUHMES cells were propagated using Nunclon™ (Nunc, Roskilde, Denmark) plastic cell culture flasks and multi-well plates that were pre-coated with 50 μg/mL poly-L-ornithine and 1 μg/mL fibronectin (Sigma-Aldrich, St. Louis, Mo., USA) in water for 3 h at 37° C. After removal of the coating solution, culture flasks were washed with sterile distilled water and air-dried.

Cells were grown at 37° C. in a humidified 95% air, 5% CO₂ atmosphere. The proliferation medium was Advanced Dulbecco's modified Eagle's medium (Advanced DMEM)/F12 containing 1× N-2 supplement (Invitrogen, Karlsruhe, Germany), 2 mM L-glutamine (Gibco, Rockville, Md., USA) and 40 ng/mL recombinant bFGF (R&D Systems). When reaching approximately 80% confluence, cells were dissociated with a 0.025% trypsin solution (Gibco, Rockville, Md., USA) and passaged at 3×10⁶ cells/flask.

To induce differentiation intoneuronal cells, 2×10⁶ LUHMES were seeded and grown into a T75 flask in proliferation medium for 48 h, then in Advanced DMEM/F12 containing 1× N-2 supplement, 2 mM L-glutamine (Gibco), 1 mM dibutyryl cAMP (Sigma-Aldrich), 1 μg/mL tetracycline (Sigma-Aldrich) and 2 ng/mL recombinant human GDNF (R&D Systems). After two days of culture in differentiation condition, LUHMES were cultured to 24-well plate for further experiments at day six.

3. LUHMES Cells Treatment

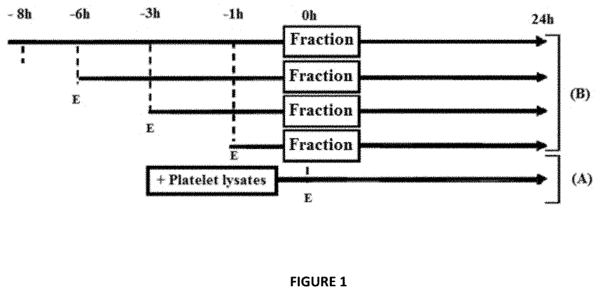

All platelet lysate preparations were used at 5% v/v and were tested against cell death induced by Erastin at 1.25 μM. Briefly, LUHMES are used as described before and the different platelet lysate fractions were added into the medium 1 h before treatment with erastin (E) or 1, 3 6 and 8 h after Erastin (FIG. 1).

4. Viability Test

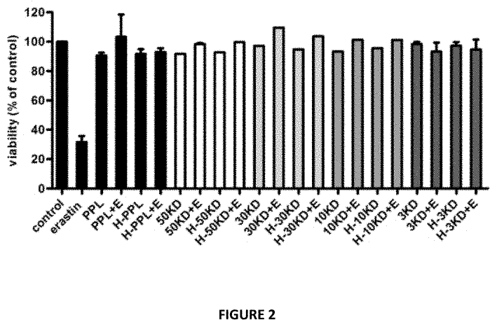

In order to quantify the neuroprotective ability of the different platelet lysate fractions, the viability of LHUMES cells was evaluated by cytometry assay in 24 wells by using propidium iodide incorporation (FIG. 2) and compared to the control or to the different platelet lysates. The cytometer used for the experiments is the CyAn™ model with a 488 nm laser (Beckman Coulter).

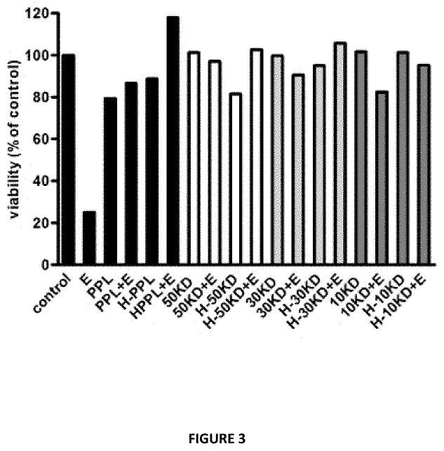

The viability was also measured by a resazurin assay performed in 96 wells at 7 days of differentiation and 24 h after treatments with 50 kDa, H-50 kDa, 30 kDa, H-30 kDa, 10 kDa, and H-10 kDa fractions. The assay is performed on the cell culture without trysinitation (FIG. 3).

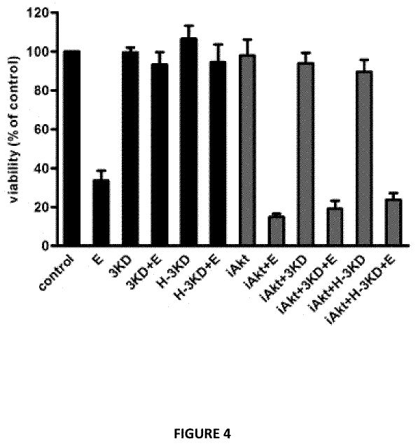

The platelet lysate H-3 kDa fraction was evaluated separately by resazurin assay in order to further determine whether the smallest fraction produced from platelet pellet lysate induces Akt signaling pathway (FIG. 4). Thus, an experiment with Akt inhibitor was performed and the inhibitor MK-2206 was added to the medium at 5 μM 1 h before exposure to platelet lysate fractions.

The treatment with platelet lysate H-3 kDa fraction was performed 1 h before exposure to Erastin and 1 h, 3 h, 6 h, and 8 h after exposure to Erastin.

5. Protein Dosage

The protein concentration was measured in different samples by a Lowry protein assay. For each sample, measurements were made in duplicate and concentrations are expressed in μg/μL.

6. Statistical Analyses

Results are expressed as the mean±standard error of the mean (SEM). Statistical analyses were performed using one-way ANOVA after checking for the normal distribution of the data. Non-parametric texts of Wilcoxon and Kruskal-Wallis were performed in case of non-normal distribution. A p value of <0.05 was considered statistically significant.

Results 1. Protein Dosage

| | Concentration | | Standard | |
|---|---|---|---|---|
| | (μg/μL) | Number | deviation | SEM |
| PPL | 17.08 | 12 | 4.20 | 1.21 |
| H-PPL | 7.92 | 14 | 2.30 | 0.61 |
| 50 kDa fraction | 0.52 | 3 | 0.21 | 0.12 |
| H-50 kDa fraction | 0.65 | 3 | 0.20 | 0.11 |
| 30 kDa fraction | 0.36 | 2 | 0.16 | 0.12 |
| H-30 kDa fraction | 0.56 | 2 | 0.12 | 0.09 |
| 10 kDa fraction | 0.41 | 2 | 0.22 | 0.16 |
| H-10 kDa fraction | 0.55 | 3 | 0.26 | 0.15 |
| 3 kDa fraction | 0.36 | 7 | 0.09 | 0.04 |
| H-3 kDa fraction | 0.40 | 13 | 0.10 | 0.03 |

2. Protective Ability on Dopaminergic Neurons
Cytometric Assay

As shown in FIG. 2, the viability of LUHMES cells treated with Erastin declines to approximately 30%. Thus, Erastin kills the control cells and this was not observed when LUHMES cells were treated concomitantly by any of platelet lysate fractions.

Indeed, none of the platelet lysate fractions had a toxic effect to the LUHMES cells. Therefore, the platelet lysate fractions protect LUHMES cells from death induced by Erastin and display strong neuroprotective effect.
Resazurin Assay

The results obtained by cytometric assay were confirmed by resazurin.

As show in FIG. 3 and FIG. 4, Erastin kills efficiently the LUHMES cells and a treatment with platelet lysate fractions according to the invention 1 h before exposure to Erastin protects LUHMES cells from death. Thus, the platelet lysate fractions are able to prevent the toxic effect of Erastin and display significant neuroprotective effect.

Moreover, FIG. 4 also shown that platelet lysate H-3 kDa fraction involves Akt signaling pathway.

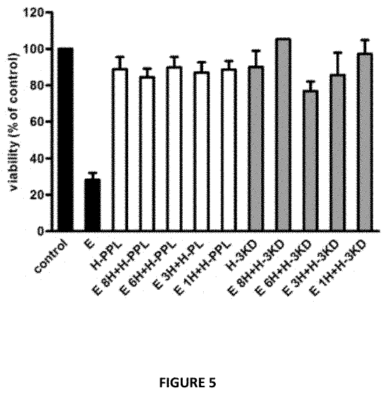

The neuroprotective effect was also tested when platelet lysate 3 H-kDa fraction was added after exposure to Erastin and the result shown (FIG. 5) that a treatment with platelet lysate H-3 kDa fraction 1 h to 8 h before exposure to Erastin still protects LUHMES cell from death.

3. Conclusion

The platelet lysate fractions according to the invention prepared with platelet pellet lysate as starting material are able to protect cells against death induced by a neurotoxic and display an effective neuroprotective effect. This result was validated with two different assays.

### Example 2—Experiments with Pooled Human Platelet Lysate (pHPL) as Starting Platelet Lysate Material

1. Preparation of the Pooled Human Platelet Lysate (pHPL) and Platelet Lysate Fractions

The pooled human platelet lysate (pHPL) is obtained from Macopharma (Tourcoing, France) under the name MultiPL'30® Human platelet lysate, reference BC0190020. A part of the pHPL was subjected to heat-treatment at 56° C. for 30 min and was purified by centrifugation (15 minutes, 10000×g, 4° C.) to obtain the so-called HT-pHPL.

An another part of the pHPL was mixed with 0.5 g/mL of glass beads (BEAD-002-1 kg of 2 mm of diameter, from Labbox) and CaCl₂ (23 mM final concentration; C4901 Calcium chloride anhydrous powder, from Sigma-Aldrich) under stirring for 1 h. It was leading to a clot formation that was removed after centrifugation (6000×g for 30 minutes at 22° C.). The supernatant was heated at 56° C. for 30 minutes and centrifuged (10000×g for 15 minutes at 4° C.) before aliquots were made and stored at −80° C. for further use. This so-obtained platelet lysate is called H-pHPL-GB.

The heat-treated. platelet lysate 3 kDa fraction was obtained from H-pHPL-GB using Amicon Ultra −0.5 ultrafiltration tubes including different cutoffs (Amicon Ultra-0.5 centrifugal Filter Devices, Millipore).

Briefly, 500 μL of H-pHPL-GB is added to the filter device inserted into the collect tube, and centrifuged at 14000×g for 30 minutes at 4° C. with a fixed angle rotor at 40°. According to the cutoff used, the filtrate or the heat-treated platelet lysate fraction, which is the lower part lower than the cutoff, is called H-3 kDa because it was obtained from a heat-treated platelet lysate.

The platelet lysate fractions were then ali quoted and stored at −80° C. for further experiments.

2. LUHMES Cells Maintenance and Differentiation

LUHMES cells were obtained and prepared as described in example 1.

3. LUHMES Cells Treatment

The H-3 kDa fraction was used at 5% v/v and was tested against cell death induced by Erastin. Briefly, LUHMES are used as described before and the H-3 kDa fraction was added into the medium 1 h before treatment with Erastin (E).

4. Viability Test

In order to quantify the neuroprotective ability of the heat-treated platelet lysate 3 kDa fraction, the viability of LHUMES cells was evaluated by cytometry assay in 24 wells by using propidium iodide incorporation and compared to the control or to the different platelet lysates. The cytometer used for the experiments is the CyAn™ model with a 488 nm laser (Beckman Coulter).

5. Protein Dosage

The protein concentration was measured in different samples by a Lowry protein assay. For each sample, measurements were made in duplicate and concentrations are expressed in μg/μL.

6. Results
protein dosage

| | Concentration | Number of | Standard | |
|---|---|---|---|---|
| | (μg/μL) | samples | deviation | SEM |
| pHPL | 19.08 | 2 | 2.47 | 1.75 |
| H-pHPL | 18.51 | 2 | 1.20 | 0.85 |
| pHPL-GB | 18.65 | 2 | 0.62 | 0.44 |
| H-pHPL-GB | 17.31 | 2 | 1.58 | 1.12 |
| H-3 kDa | 0.09 | 2 | 0.02 | 0.02 |

Protective ability on dopaminergic neurons

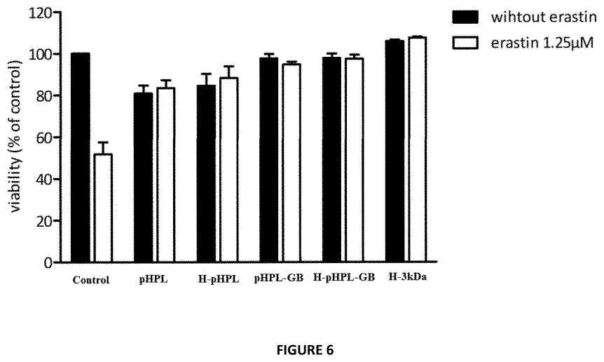

As shown in FIG. 6, the viability of LUHMES cells treated with Erastin declines to approximately 50%. Thus, Erastin effectively kills the control cells and this was not observed when LUHMES cells were treated concomitantly the H-3 kDa fraction.

Therefore, the H-3 kDa fraction protects LUHMES cells from death by ferroptosis and display strong neuroprotective effect.

This example exhibits the potential of the heat-treated platelet lysate 3 kDa fraction according to the invention. Moreover, this fraction was obtained from a pooled human platelet lysate which offers a substantially higher safety margin than standard human platelet lysates suspended in plasma. Thus, this H-3 kDa fraction is more suitable and more efficient for use in biotherapy, especially through brain administration.

### Example 3—In Vivo Tests

All experiments were carried out in accordance with the “Principles of Laboratory Animal Care” (NIH publication 86-23, revised in 1985) and the current French and European Union legislative and regulatory framework on animal experimentation (The Council of the European Communities Directive 86/609).

The mice enrolled were FVB-Tg (Sod1*G86R) M1Jwg/J mice from JAX laboratories. Animals were group-housed (10 per cage) in a temperature-controlled room (22±2° C.) with a 12/12-hour light/dark cycle. Food and water were feed ad-libitum. After reception, the animals had a 7-day habituation period with no handling. Breeding was realized in SOPF facility and genotyping is performed by qPCR (from tail biopsy). Animal are identified with earrings.

1. Experimental Protocol

Mice were handled and weighted at the age of 60 days and they were evaluated twice a week (i.e. body weight and neuroscore) from the age of 67 days to their death.

a. Neuroscore
walking (0=ok, 1=a single hindpaw alteration, 2=two hindpaws alteration),
tail suspension test (0=ok, 1=a single hindpaw retractation, 2=two hindpaws retractation)
paralysis (0=no, 1=yes)
Kyphosis (0=no, 1=yes)

Max score=6 (moribund score)

b. Treatment

From 75 days to death, different platelet lysate preparations versus vehicle are administrated three times a week (preparations 1 and 2) and six times a week (preparation 2 only) in SOD1m-FVB and WT-FVB males and females. The SOD1m-FVB mice are transgenic mice overexpressing mutant forms of the copper/zinc superoxide dismutase gene. The dose of preparation administrated was 20 μL intra nasally (i.n.).

c. Platelet Lysate Tested

Three platelet lysate preparations have been tested and are described hereafter.

Preparation 1: H-PPL as described in example 1, section 1 and diluted at 50% in PBS (H-PPL diluted).

Preparation 2: heat-treated platelet lysate 3 kDa fraction (H-3 kDa fraction) prepared from H-PPL as described in example 1, section 1.

d. Experimental Groups

Eight groups have been constituted as follow:

Males WT-FVB+vehicle

Males WT-FVB+preparations (H-PPL, H-PPL diluted or H-3 kDa fraction)

Males SOD1m-FVB+vehicle

Males SOD1m-FVB+preparation (H-PPL, H-PPL diluted or H-3 kDa fraction)

Females WT-FVB+vehicle

Females WT-FVB+preparation (H-PPL, H-PPL diluted or H-3 kDa fraction)

Females SOD1m-FVB+vehicle

Females SOD1m-FVB+preparation (H-PPL, H-PPL diluted or H-3 kDa fraction)

2. Results

a. Preparation 1: H-PPL Diluted
Body weight

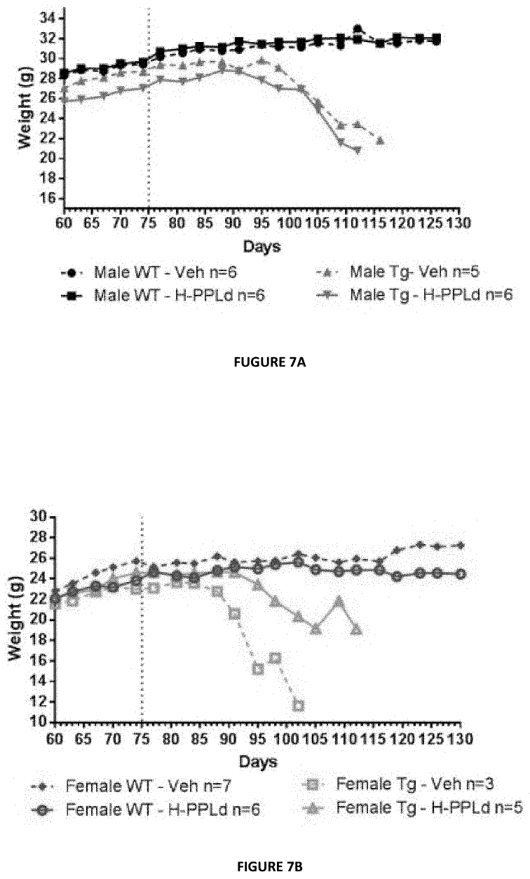

As shown in FIGS. 7A and 7B, treatments with H-PPL diluted had no effect in WT males and females.

Regarding to the body weight decline, no difference was observed in Tg males. Nevertheless, in Tg females, H-PPL diluted treatment maintains the body weight at the beginning level up to Day 95 with almost 10 days of difference with vehicle group and also delays the pre-mortem body weight from 10 days (D102-D112).
Survival curve

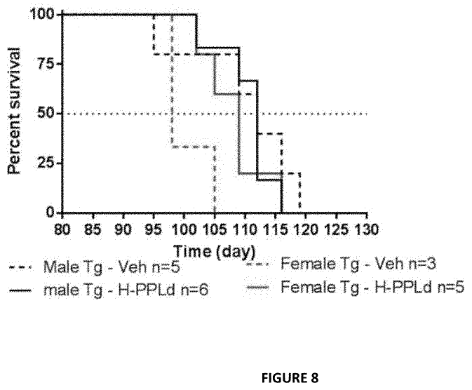

In accordance with the body weight, in Tg females treated with H-PPL diluted had an extended survival from D105 to D116 (11 days more). This difference was not observed in Tg Male mice between the vehicle and treated group. (FIG. 8)

b. Preparation 2: H-3 kDa Fraction

Three Times a Week Administration:
Body weight

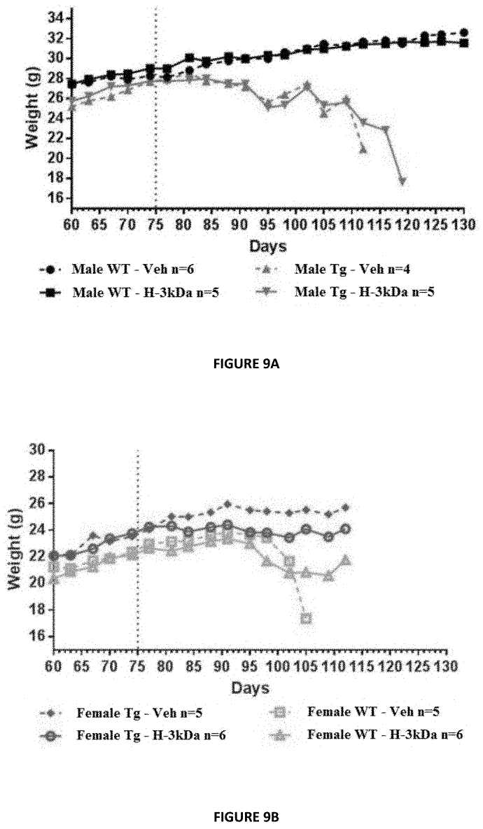

As shown in FIG. 9A, in the WT mice, H-3 kDa fraction had no effect in male body weight. Contrary to the males, in females a slight decrease is observed shortly after the beginning of the treatment (D81) and throughout its duration.

In both Tg males and females, no improvement is observed in body weight loss initiation (compare to Tg mice with vehicle) but, H-3 kDa fraction induced an important delay in the pre-mortem body weight of 21 days for females mice (from D105 to D126) and of 7 days for males mice (from D112 to D119) (FIG. 9B).
Survival curve

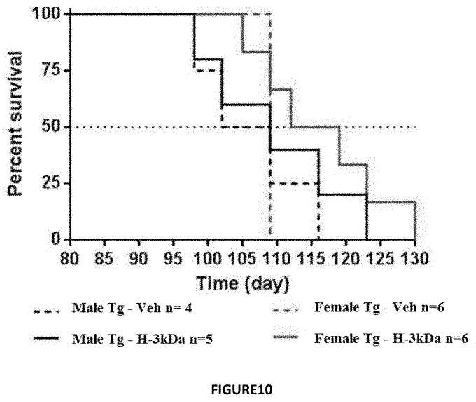

As show in FIG. 10 and in accordance with the delay observed in the post-mortem body weight, a treatment with H-3 kDa fraction extended survival time up to 21 days for Tg females (D109 to D130) and up to 7 days for Tg males (D116 to D123).

Six Times a Week Administration:
Body weight

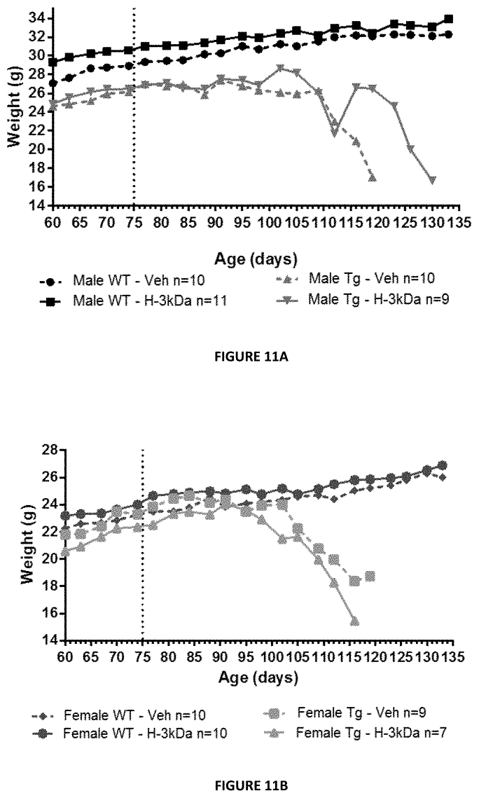

As shown in FIGS. 11A and 11B, in the WT mice, H-3 kDa fraction had no effect in male and female body weight.

In both Tg males and females, no improvement is observed in body weight loss initiation (compare to Tg mice with vehicle) but, H-3 kDa fraction induced a delay in the pre-mortem body weight of 11 days for males mice (from D119 to D130). This effect is not observed in Tg Female.
Survival curve

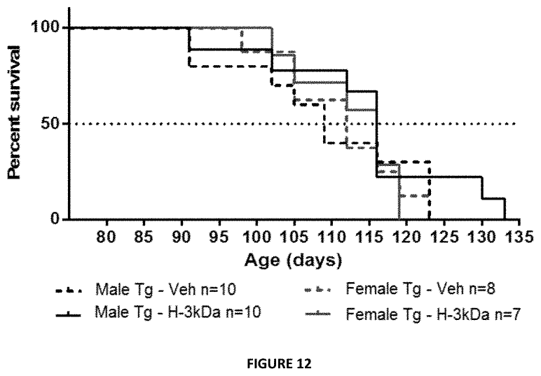

As show in FIG. 12, and in accordance with the delay observed in the post-mortem body weight, a treatment with H-3 kDa fraction extended survival time up to 21 days for Tg females (D109 to D130) and up to 7 days for Tg males (D116 to D123).

3. Conclusion

No irritation was observed with H-PPL diluted and H-3 kDa treatments.

Regarding the survival curves obtained with different preparation, it can be observed that the effect depends on animal gender.

But, the main conclusion of this in vivo test is that H-3 kDa has an excellent safety profile and a great efficacy with a classical sex-dose related effect. Indeed, treatment with H-3 kDa extended survival by 7 days in Tg males mice and provided an increase of about 21 days in Tg females mice (90% of improvement as compared with controls) and of about 10 days in Tg males mice (48% of improvement as compared with controls). Those results show the potential of the heat-treated platelet lysate 3 kDa fraction according to the invention in order to induce neuroprotective ability.

### REFERENCES

1. Gonzalez-Aparicio R, Flores J A, Fernandez-Espejo E. Antiparkinsonian trophic action of glial cell line-derived neurotrophic factor and transforming growth factor beta 1 is enhanced after co- infusion in rats. Experimental Neurology 2010; 226: 136-47.
2. Golebiewska E M, Poole A W. Platelet secretion: From haemostasis to wound healing and beyond. Blood Rev 2014.
3. Burnouf T, Goubran H A, Chen T M, et al. Blood-derived biomaterials and, platelet growth factors in regenerative medicine. Blood Rev 2013; 27: 77-89.
4. Burnouf T, Strunk D, Koh M, et al. Human platelet lysate: replacing fetal bovine serum as a gold standard for human cell propagation? Biomaterials 2016; 76: 371-87.
5. Hayon Y, Dashevsky O, Shai E, et al. Platelet lysates stimulate angiogenesis, neurogenesis and neuroprotection after stroke. Thromb Haemost 2013; 110: 323-30.
6. Yael Hayon; Olga Dashevsky; Ela Shai; David Varon; Ronen R. Leker Platelet lysates stimulate angiogenesis, neurogenesis and neuroprotection after stroke. Thromb Haemost 2013; 110: 323-330
7. Shih D T B, Burnouf T. Human blood platelet growth factors supplements for ex vivo stem cell expansion (invited review). New Biotechnology, 2015: 32; 199-211.
8. Tsu-Bi Shih D, Burnouf T. Preparation, quality criteria, and properties of human blood platelet lysate supplements for ex vivo stem cell expansion. New Biotechnology 2015; vol 32, number 1.
9. Victor E. Santo, Manuela E. Gomes, Joao F. Mano and Rui L; Reis. Chitosanchondrotin sulphate nanoparticles for controlled delivery of platelet lysates in bone regenerative medicine. Journal of Tissue Engineering and Regenerative Medicine. December 2012, vol.6, issue S3, pages s47-s59.
10. Scholz D, Poltl D, Genewsky A, et al. Rapid, complete and large-scale generation of post-mitotic neurons from the human LUHMES cell line. J Neurochem 2011; 119: 957-71.

1. A process for preparing a platelet lysate fraction, said process comprising the steps of:
1) providing a platelet lysate, and
2) collecting by ultrafiltration from said platelet lysate a fraction wherein the components exhibit a maximum molecular weight of 100 kDa.

2. The process according to claim 1, wherein the platelet lysate provided in step 1) is a platelet pellet lysate or a pooled human platelet lysate.

3. The process according to claim 1, wherein the collection step is a fractionation carried out by ultrafiltration.

4. The process according to claim 1, wherein the following steps are performed after step 1) and before step 2):
a step of heat-treating the platelet lysate at a temperature of about 50° C. to about 70° C. during 15 minutes to 45 minutes, and
a step of centrifuging said heat-treated platelet lysate and keeping the supernatant for the collection step.

5. A platelet lysate fraction characterized in that the components contained therein exhibit a maximum molecular weight of 20 kDa.

6. The platelet lysate fraction according to claim 5, characterized in that the components contained therein exhibit a maximum molecular weight of 10 kDa or of 3 kDa, and preferably a maximum molecular weight of 3 kDa.

7. The platelet lysate fraction according to claim 5, having a protein content of less than 1.5 μg/μL, preferably less than 1.0 μg/μL and more preferably less than 0.70 μg/μL.

8. The platelet lysate fraction according to claim 7, wherein the protein content is of about 0.05 μg/μL to about 0.30 μg/μL, and more particularly of about 0.05 μg/μL to about 0.1 μg/μL.

9. The platelet lysate according to claim 8, wherein said fraction is free of fibrinogen.

10. A pharmaceutical composition, comprising the platelet lysate fraction according to claim 5 and at least one pharmaceutically acceptable carrier, diluent, excipient and/or adjuvant.

11. A method for the treatment of disorders of the central nervous system, comprising the step of administering a platelet lysate fraction according to claim 5 to a patient in need thereof.

12. The method according to claim 11, wherein the disorders of central nervous system are selected from neurodegenerative disorders, neurovascular disorders, neuroinflammatory disorders, neurodevelopmental disorders and cerebral insults.

13. The method according to claim 12, wherein the disorders of central nervous system is a neurodegenerative disorder selected from multiple sclerosis (MS), Parkinson's disease (PD), Huntington's disease (HD), Amyotrophic lateral sclerosis (ALS), stroke, age-related macular degeneration (AMD), Alzheimer's disease (AD), vascular dementia, frontotemporal dementia, semantic dementia and dementia with Lewy bodies and preferably selected from Parkinson's disease and Amyotrophic lateral sclerosis.

14. The method according to claim 13, wherein the neurodegenerative disorders are selected from Parkinson's disease.

15. The method according to claim 14, wherein the disorder is a cerebral insult selected from hypoxia or traumatic brain injury.

16. The method according to claim 10, wherein the platelet lysate fraction is administrated by intranasal, intrathecal, intraocular or intra cerebroventricular route.

17. The method according to claim 16, wherein the platelet lysate fraction is administrated by intra cerebroventricular route, preferably closed to the intraventricular foramen and more preferably into the third ventricle.

18. The method according to claim 17, wherein said platelet lysate fraction is adapted to be administered with a pump.
